(19) United States
(12) Patent Application Publication
(10) Pub. No.: US 2008/0108979 A1
(43) Pub. Date: May 8, 2008

(54) Flush Tip Illuminating Laser Probe Treatment Apparatus

(76) Inventors: William Telfair, San Jose, CA (US); Charles Bilek, Campbell, CA (US)

Correspondence Address: HELLER EHRMAN LLP, 275 MIDDLEFIELD ROAD, MENLO PARK, CA, 94025-3506, US

(21) Appl. No.: 11/556,504

(22) Filed: Nov. 3, 2006

(51) Int. Cl. A61B 18/18

(52) U.S. Cl. 606/4

(57) ABSTRACT

A treatment apparatus has a probe needle with a distal end, a laser fiber and a plurality of illumination fibers. The laser fiber and the plurality of illumination fibers are substantially flush with the distal end of the probe needle. The illumination fibers of the plurality of illumination fibers have a numerical aperture greater than 0.55.

### BACKGROUND OF THE INVENTION

1. Field of the Invention

This invention relates generally to an illuminating probe treatment apparatus, and more particularly to a illuminating probe treatment apparatus that has a large illumination field with a smaller treatment area, and a substantially smooth surface which does not catch on tissue.

2. Description of the Related Art

Ophthalmic surgeons have used straight endo photocoagulator probe instruments to perform laser surgery on the retina in the back of the globe for many years. Examples of these probes are described in U.S. Pat. Nos. 4,537,193 and 4,865,029.

Curved versions of these probes were introduced to allow the surgeon to reach more distant regions of the retina without distorting the access port. These probes typically are bent to either 30 degrees or to 45 degrees. They are typically used without a cannula on the larger gauge treatments (20 gauge) where a suture is required to seal the wound after the surgery.

An alternative to the curved probes above are probes called stepped angled probes as described in U.S. patent application Ser. No. 11/205,629—“Directional Probe Treatment Apparatus”. These needles are ground down to smaller ODs (or stepped down to smaller gauges) at the distal end so that the curved portion will go through a cannula. This allows a curved needle to go through the cannula and still treat over a large angular range.

Improvements in the probes were introduced by combining multiple functions into a single instrument rather than requiring multiple probes and frequent removal and insertion of these probes. One example of this is combining aspiration with laser treatment as described in U.S. Pat. No. 5,318,560.

Another example is combining illumination with the laser treatment into a single probe as described in U.S. Pat. Nos. 5,323,766 and 5,356,407. These probes have the disadvantages of the illumination area being the same or similar size as the treatment area. The surgeon needs to observe a larger area to confirm that the treatment is the proper location. Hence, to use these probes, the doctor would pull the probe back to illuminate a large area and then push it up to the treatment area for laser treatment. This involves a lot of manipulation of the probe with the potential for occasional incidents of contacting the retina by mistake.

The bayonet style illuminating probe was introduced to provide a wider illumination field while the laser fiber was close to the treatment area. The bayonet style means that the laser fiber protrudes beyond the illumination fiber or fibers. Thus it is closer to the retina and will treat a smaller area than the illuminated field. However, with the laser fiber protruding, it can catch on tissue and tear or damage the tissue or, even worse, it can break off and leave fragments in the eye. This can occur either during introduction of the probe into the eye through the globe wall or during treatment of the retina. In addition, with the treatment fiber protruding, it can cast a shadow to one side of the illumination field.

One solution to the tissue damage issue is to add a soft tip cover onto the probe. Such a probe is described in U.S. Pat. No. 5,441,496 and No. 5,603,710. This soft tip protects the tissue and fiber from breakage and damage issues, yet allows some flexibility for the fiber to protrude beyond the end of the needle.

The illuminating probes all have a bifurcated design with the laser fiber going to the laser connection and the illumination fibers/fibers going to the light source connection When they are connected to the light source and the light source is turned on, the illumination connector gets very hot. We have measured up to 76 degrees C. on these connectors. Physicians turn them off and wait for them to cool down before disconnecting them. However, in an emergency, they could easily burn themselves on this connector.

Additional probes called directional probes have been developed to allow the physician to adjust the probe fiber bend angle, so that he/she can treat anywhere in the retina from center to far periphery. Examples of these probes are described in U.S. Pat. Nos. 6,572,608 and 6,984,230. Another example of this design called the adjustable or intuitive probe is US patent application 2005/0154379 A1. None of these have illumination, because they can't fit the illumination fibers into the package with all the other components.

There is a need for an illuminating probe that: 1) doesn't have a shadow, 2) has a large illumination field with a smaller treatment area, 3) has a smooth surface that doesn't catch on tissue, 4) has a bright uniform illumination, 5) can be constructed into a small gauge needle, 6) can be constructed into curved and/or directional or intuitive probes, and 7) has an illumination connector design which can be handled at all times.

### SUMMARY

Accordingly, an object of the present invention is to provide an illuminating probe treatment apparatus that does not have a shadow.

Another object of the present invention is to provide an illuminating probe treatment apparatus that has a large illumination field with a smaller treatment area.

Yet another object of the present invention is to provide an illuminating probe treatment apparatus that has a substantially smooth surface which does not catch on tissue.

Still a further object of the present invention is to provide an illuminating probe treatment apparatus that provides bright, uniform illumination.

A further object of the present invention is to provide an illuminating probe treatment apparatus that is constructed into a small gauge needle.

Another object of the present invention is to provide an illuminating probe treatment apparatus that has a needle which is at least partially curved or directional.

In one embodiment of the present invention, a treatment apparatus that has a probe needle with a distal end, a laser fiber and a plurality of illumination fibers. The laser fiber and the plurality of illumination fibers are substantially flush with the distal end of the probe needle. The illumination fibers of the plurality of illumination fibers have a numerical aperture greater than 0.55.

In another embodiment of the present invention, a treatment apparatus has a probe needle with a distal end, a laser fiber and a plurality of illumination fibers. The laser fiber and the plurality of illumination fibers are substantially flush with the distal end of the probe needle. The plurality of illuminated fibers provide an illumination area that is at least 75 times larger than a laser treatment area provided by the laser fiber.

In another embodiment of the present invention, a treatment apparatus has a probe needle with a distal end, a laser fiber, and a plurality of illumination fibers. The laser fiber and the plurality of illumination fibers are substantially flush with the distal end of the probe needle. The plurality of illumination fibers have a packing density greater than 45% of the area of the distal end of the needle.

### BRIEF DESCRIPTION OF THE DRAWINGS

FIG. 1 illustrates one embodiment of a flush tip illumination probe of the present invention.

FIG. 2 illustrates the relationship of the different diameters of the probe needle, laser fiber and illumination fibers of the FIG. 1 embodiment, and shows the packing density of the illumination fibers.

FIG. 3 illustrates a angled or curved embodiment of the present invention.

FIG. 4 illustrates a stepped angled or stepped curved embodiment of the present invention.

FIG. 5 illustrates an adjustable/intuitive or directional embodiment of the present invention.

FIG. 6 illustrates a illumination connector with thermal protection embodiment of the present invention.

### DETAILED DESCRIPTION

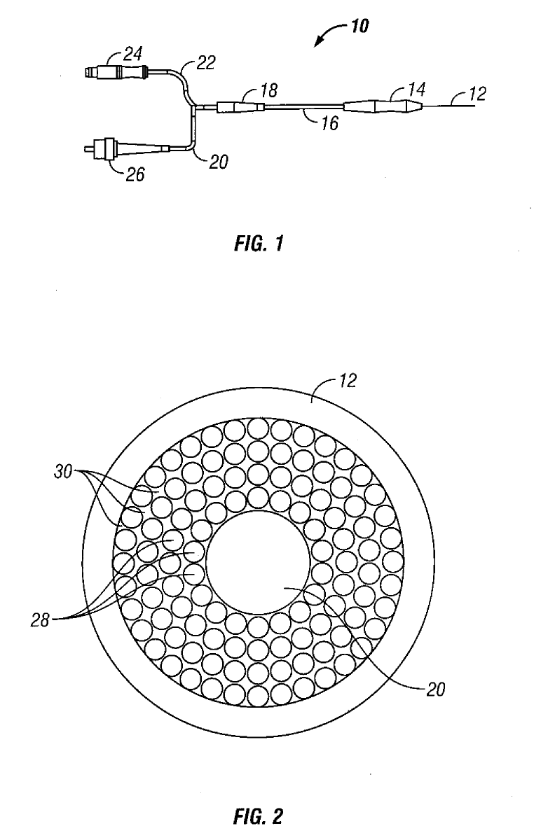

Referring to FIG. 1, one embodiment of the present invention is a flush tip illuminating probe, generally denoted as 10, that has a probe needle 12 and a handle (or handpiece) 14. The needle 12 has a diameter which is typically between 20 and 25 gauge. 20 to 25 gauge is the important range in ophthalmic surgery. It will be appreciated, however, that the probe 10 can be used for other tissue sites in the body. Dimensions much smaller than 25 gauge, higher gauge numbers, such as 26 or 27 gauge are less important for ophthalmic applications due to incompatibility with existing support instrumentation and the increasing difficulty of coupling therapeutic modalities such as laser, electrosurgery, diathermy, and the like.

The probe 10 further includes a jacketed fiber bundle 16. This fiber bundle 16 is bifurcated at a union piece 18 into a laser fiber 20 and an illumination fiber bundle 22. In one embodiment, the laser fiber 20 and the plurality of illumination fibers 22 with the distal end of the probe needle 12 are configured to provide a smooth surface that doesn't catch on tissue and are substantially flush with the proximal end of the probe needle 12.

The illumination fiber bundle 22 is terminated in a connector 24, at a proximal end, which can be plugged into an illumination source, either directly or with an adaptor (not shown). The laser fiber is terminated into a standard SMA 905 style fiberoptic connector 26 or other style of connector such as a 906 style or ST style connector.

If the illumination source produces sufficient wattage of light, the illumination connector 24, at the distal end of the probe 10, can get hot, especially if it is left plugged into the source for more than 5 to 10 minutes. Temperatures well above 50 degrees centigrade on this metal connector have been measured. The probe 10 can incorporate a thermal cover or sleeve 60 to cover the metal surface of the illumination connector as shown in FIG. 6. The sleeve 60 can be a high temperature plastic which conducts much less heat and keeps the operator from burning himself or herself when unplugging the connector. The sleeve can also be made from an insulating coating material including but not limited to, fiberglass, foam, ceramic using deposition techniques and the like.

The fibers of fiber bundle 22 are glued into each of the connectors and then polished to be flush with the end of the connector. The fibers are also glued into the needle 12. At this distal end, the laser fiber 20 is much larger and is fed first through the needle 12. The individual fibers from the illumination fiber bundle 22 are then fed through the needle 12.

Referring now to FIG. 2, a cross section of the needle 12 illustrates that in one embodiment the larger laser fiber 20 is in the center, although this is not always true and not necessary, due to one embodiment of an assembly process. The individual illumination fibers 28 crowd into the available space until the inner diameter (ID) of the needle 12 is filled. From typical dimensions of the outer diameter (OD) of 140 microns for the laser fiber, 50 microns OD for the illumination fibers and 430 micron ID for a 25 gauge needle 12, 35 to 45 illumination fibers 28 can be packed into the available space. These are fixed in place with glue 30 once they have all been fed through the needle 12.

Although the wall of the needle 12 is thin, by way of illustration and without limitation such as 31 to 120 microns, and the needle 12 is quite flexible and fragile when empty, after the glass fibers are glued into the needle 12, the assembly is much stiffer and much less fragile. This packing process helps improve the quality of the assembled product.

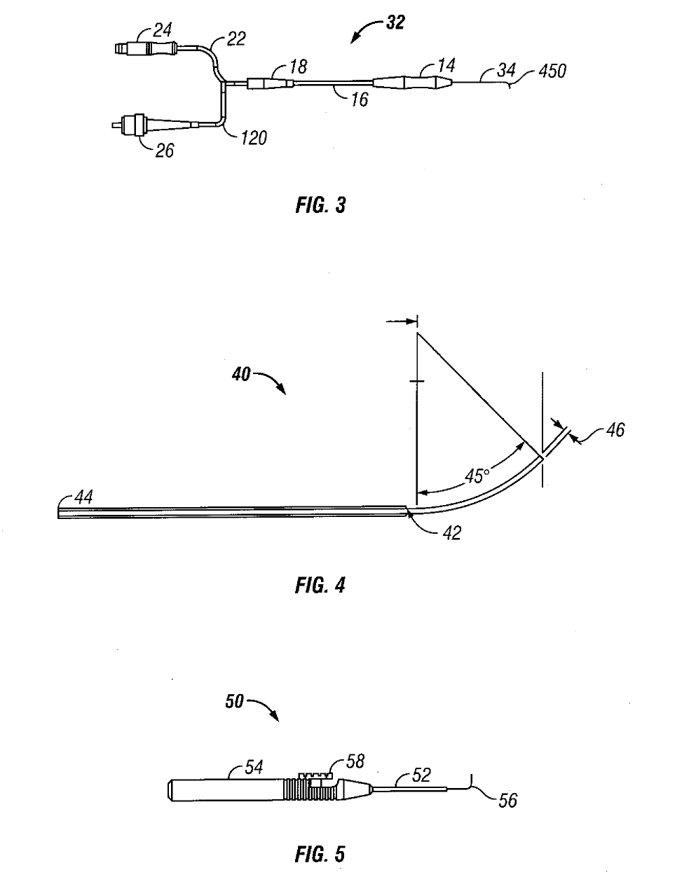

Referring to FIG. 3, another embodiment of the present invention is an angled (or curved) flush tip illuminating probe 32, that has a probe needle 34, which is angled. The rest of the probe 32 is the same as the straight needle probe 10 illustrated in FIG. 1. The angled needle 34 is typically curved to an angle of 30 to 45 degrees. The radius of curvature of the needle 34 is large compared to the needle diameter so that there shall be no kinks in the needle 34, the ID of the needle 34 is unchanged and the fibers can be fed through the needle 34 in the same manner as they are in the straight needle 12. The needle can be curved prior to loading the fibers or the assembly can be bent after the fibers are installed and glued in place.

Referring to FIG. 4, another embodiment of the present invention is a stepped angled probe 39. The needle 40 of the probe 39 has a similar design to the angled probe in FIG. 3, except that the outer diameter (OD) of this needle 40 is stepped down at location 42. This needle tip is stepped down from the starting gauge 44 to a smaller gauge 46 (larger gauge number). The example illustrated in FIG. 4 is stepped from 23 gauge at the proximal end 44 to 27 gauge at the distal end 46. After this tip is stepped down, it is bent such that the curved part can go through a 23 gauge cannula. Additional description can be found in U.S. patent application US-2006-0041291-A1, incorporated herein by reference.

The stepped angled probe needle tip, with the laser fiber 20 and illumination fibers 22, is typically curved to 30 degrees or 45 degrees. In the FIG. 4 embodiment, it is curved to 45 degrees.

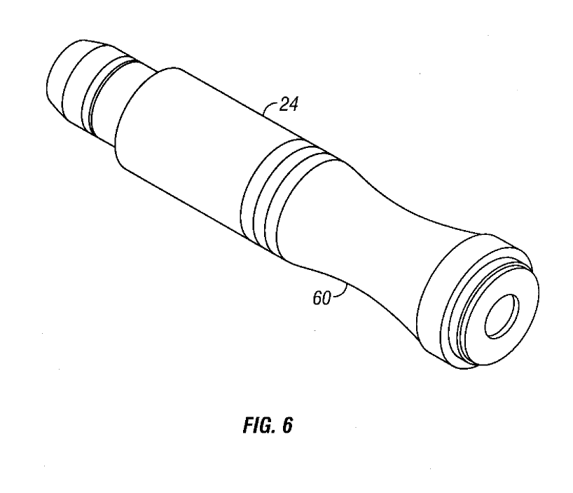

Referring to FIG. 5, another embodiment of the present invention is an adjustable/intuitive flush tip illuminating probe 50, which has a different design for the needle 52 and a different design for the handle 54. This needle 52 has the laser fiber 20 and the illumination fibers 30 wrapped in a memory metal 56, which can be forced into a straight line within the steel needle 52, but will take another shape from the memory metal when protruding from the needle 52. In this example, the shape is a 90 degree bend. Additional information relative to this embodiment is described in U.S. Pat. No. 6,984,230 or U.S. patent application 2005/0154379A1, incorporated herein by reference. The thumb slide tab 58 is attached to the memory metal and fibers and is used to slide the fibers out of the needle 52 to the desired angle for treatment.

An embodiment similar to FIG. 5 can also be a directional probe as described in U.S. Pat. No. 6,572,608 (the “'608 Patent”), incorporated herein by reference The directional probe of the '608 patent has a memory metal, a hollow memory metal with a fiber positioned in the center but does not have illumination. This embodiment is different from the previous one in that the needle is affixed to the thumb slide tab 58 and moved in and out. When the needle is pulled back the fiber and memory metal sleeve are exposed and become curved—taking the shape of the memory metal.

The probes of the present invention have small diameter illumination fibers. In various embodiments, the illumination fibers have core diameters, excluding the cladding of 30-75 microns, 40-50 microns and 45 microns. This allows many fibers to be packed into available space with very little space wasted. In one embodiment of the present invention, using fibers with 90% core and 10% cladding, the packing density of the fibers is about 50-60%. The fibers in this embodiment have diameters in the range of 30-75 microns. Packing density is defined as total fiber core area divided by the total area in %. The packing density for previous probes with one illumination fiber the same size as the laser fiber is 35%. The packing density for previous probes with multiple illumination fibers is 33% to 41%.

With the present invention, this dense packing collects more light from the source and delivers more light to the treatment site. The smaller diameter also allows the fibers to be packed into smaller spaces such as the adjustable probe, where the ID of the memory metal is smaller than the needles used previously for illumination probes.

Another advantage of the present invention is the high numerical aperture (NA) of the individual illumination fibers. This property of these fibers allows collection of more light and higher NA light from the source yielding a higher efficiency of optical transfer. This light is transmitted and delivered to the treatment site, illuminating a larger area with more optical power. Since the illumination fibers are more efficient, the light source does not need to be turned up as high and will have a longer lifetime. In one embodiment, the illumination fibers have an NA of 0.65 to 0.75.

This larger NA allows the illumination fibers to be flush with the laser fiber and still deliver a wide illumination field for the doctor to see the treatment site. The laser fiber doesn't need to protrude beyond the illumination fibers and the needle end. This eliminates the dangers of a laser fiber catching on tissue, tearing or damaging tissue or breaking off and being left in the eye.

This high NA illumination fiber allows the multiple types of probe designs described in FIGS. 1, 2, 3, 4, & 5. The spot size for these probes is shown in Table 1. This table shows the illuminated spot area for previous flush-type probes, bayonet-type probes and for the flush-tip probes of this invention versus the distance that the probe tip is from the treatment surface (presumably the retina in ophthalmic treatments). For example, with the laser fiber 3 mm from the retina, the area illuminated with this new probe is over 31 mm² compared to less than 8 mm² for the previous flush-type probe and to less than 22 mm² for the bayonet style probe. This spot size is almost 50% larger than the area of previous bayonet probes without the safety concerns, the cost of construction, or limitations in design flexibility.

In addition, Table 1 compares the laser spot size to the illumination area. This is an important comparison for the physician, since he/she needs to be able to see a much larger area around the treatment site to insure proper centration and treatment. For the same example of 3 mm from the retina, the probe of the present invention is more than 80 times the laser treatment spot size.

TABLE 1

| | | Previous flush | Bayonet | Invention |
|---|---|---|---|---|
| | | Illumination | Illumination | Illumination |
| Distance | Laser spot | spot | spot | spot |
| from retina | area (mm²) | area (mm²) | area (mm²) | area (mm²) |
| 2 mm | 0.198 | 3.733 | 14.930 | 14.862 |
| 2.5 mm | 0.286 | 5.350 | 18.020 | 22.396 |
| 3 mm | 0.389 | 7.306 | 21.483 | 31.371 |
| 3.5 mm | 0.506 | 9.539 | 25.250 | 41.854 |
| 4 mm | 0.643 | 12.069 | 29.225 | 53.716 |

The foregoing description of embodiments of the present invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A treatment apparatus, comprising:
a probe needle at the distal end of the apparatus;
a laser fiber;
a plurality of illumination fibers, the laser fiber and the plurality of illumination fibers being substantially flush with the distal end of the probe needle; and
wherein the illumination fibers of the plurality of illumination fibers have an numerical aperture greater than 0.55.

2. The apparatus of claim 1, further comprising:
a cannula adapted to receive the probe needle.

3. The apparatus of claim 1, wherein the probe needle is inserted into a puncture made by a puncturing device.

4. The apparatus of claim 1, wherein the probe needle has an outer diameter of at least one of, 25 gauge, 23 gauge and 20 gauge.

5. The apparatus of claim 1, wherein at least a portion of the probe needle has a curved or angled geometry.

6. The apparatus of claim 1, wherein at least a portion of the probe needle has a stepped angled or curved geometry.

7. The apparatus of claim 1, wherein at least a portion of the probe needle is a directional or adjustable needle.

8. The apparatus of claim 1, further comprising:
an illumination connector coupled to the apparatus proximal end, the illumination connector being at least partially covered with a thermal insulation sleeve.

9. A treatment apparatus, comprising:
a probe needle at the distal end of the apparatus;
a laser fiber;
a plurality of illumination fibers, the laser fiber and the plurality of illumination fibers being substantially flush with the distal end of the probe needle; and
wherein the plurality of illuminated fibers provide an illumination area that is at least 75 times larger than the laser treatment area provided by the laser fiber.

10. The apparatus of claim 9, further comprising:
a cannula adapted to receive the probe needle.

11. The apparatus of claim 9, wherein the probe needle is inserted into a puncture made by a puncturing device.

12. The apparatus of claim 9, wherein the probe needle has an outer diameter of at least one of, 25 gauge, 23 gauge and 20 gauge.

13. The apparatus of claim 9, wherein at least a portion of the probe needle has a curved or angled geometry.

14. The apparatus of claim 9, wherein at least a portion of the probe needle has a stepped angled or curved geometry.

15. The apparatus of claim 9, wherein at least a portion of the probe needle is a directional or adjustable needle.

16. The apparatus of claim 9, further comprising:
an illumination connector coupled to the apparatus proximal end, the illumination connector being at least partially covered with a thermal insulation sleeve.

17. A treatment apparatus, comprising:
a probe needle at the distal end of the apparatus;
a laser fiber;
a plurality of illumination fibers, the laser fiber and the plurality of illumination fibers being substantially flush with the distal end of the probe needle; and
wherein plurality of illumination fibers have a packing density over 45%.

18. The apparatus of claim 17, further comprising:
a cannula adapted to receive the probe needle.

19. The apparatus of claim 17, wherein the probe needle is inserted into a puncture made by a puncturing device.

20. The apparatus of claim 17, wherein the probe needle has an outer diameter of at least one of, 25 gauge, 23 gauge and 20 gauge.

21. The apparatus of claim 17, wherein at least a portion of the probe needle has a curved or angled geometry.

22. The apparatus of claim 17, wherein at least a portion of the probe needle has a stepped angled or curved geometry.

23. The apparatus of claim 17, wherein at least a portion of the probe needle is a directional or adjustable needle.

24. The apparatus of claim 17, further comprising:
an illumination connector coupled to the apparatus proximal end, the illumination connector being at least partially covered with a thermal insulation sleeve.
